(19) United States
(12) Patent Application Publication
(10) Pub. No.: US 2026/0130870 A1
(43) Pub. Date: May 14, 2026

(54) PHENYLEPHRINE LIQUID FORMULATIONS

(71) Applicants: FRESENIUS KABI AUSTRIA GMBH, Graz (AT); FRESENIUS KABI USA, LLC, Lake Zurich, IL (US)

(72) Inventors: Andreas UELLEN, Graz (AT); Matthias STEURER, Graz (AT); Eva TROTSCHA, Graz (AT); Daniel SCHENCK, Lake Zurich, IL (US)

(73) Assignees: FRESENIUS KABI AUSTRIA GMBH, Graz (AT); FRESENIUS KABI USA, LLC, Lake Zurich, IL (US)

(21) Appl. No.: 19/119,671

(22) PCT Filed: Oct. 19, 2023
(86) PCT No.: PCT/US2023/035490

§ 371 Date: Apr. 9, 2025

Related U.S. Application Data
(60) Provisional application No. 63/380,100, filed on Oct. 19, 2022.

(51) Int. Cl. A61K 31/137; A61J 1/14; A61K 9/08; A61K 47/02; A61M 5/178

(52) U.S. Cl. CPC A61K 31/137; A61J 1/1468; A61K 9/08; A61K 47/02; A61M 5/178

(57) ABSTRACT

The present invention provides a formulation that includes a therapeutically effective amount of phenylephrine or a pharmaceutically acceptable salt thereof, and a method for preparing the formulation. In some embodiments, the formulation includes a tonicity agent, a citrate buffer, water, and has an initial pH of from 5.6 to 6.0. In other embodiments, the formulation includes a tonicity agent, a citrate buffer, an antioxidant, water, and has an initial pH of from 3.6 to 4.8. The formulation of the invention is sterile, stable, ready-to-administer and packaged into a polymeric syringe.

### CROSS-REFERENCE TO RELATED APPLICATIONS

This patent application claims the benefit of U.S. Provisional Application No. 63/380,100, filed Oct. 19, 2022, the disclosure of which is incorporated herein by reference in its entirety for all purposes.

### BACKGROUND OF THE INVENTION

Phenylephrine is an alpha-1 adrenergic receptor agonist that functions as a vasoconstrictor. Phenylephrine can be administered orally, intranasally, topically to the eye, or by injection. Phenylephrine, also referred to as 3-[(1R)-1-hydroxy-2-(methylamino)ethyl]phenol or (−)-m-hydroxy-α-[(methylamino)methyl]benzyl alcohol, has a molecular weight of approximately 167.2 and the following chemical structure:

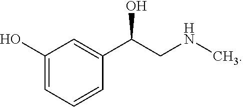 [SMILES: CNC[C@H](O)c1cccc(O)c1]

In medicine, injections of phenylephrine, or a pharmaceutically acceptable salt thereof, are indicated for the treatment of clinically important hypotension resulting primarily from vasodilation in the setting of anesthesia. VAZCULEP™ injection is available in the United States as a sterile solution in a glass vial containing 10 mg/mL phenylephrine hydrochloride, 3.5 mg/mL sodium chloride, 4 mg/mL sodium citrate dihydrate, 1 mg/mL citric acid monohydrate, and 2 mg/mL sodium metabisulfite in water for injection with sodium hydroxide and/or hydrochloric acid, if necessary, to provide a pH of 3.5 to 5.5. Generic pharmaceutical vial products are available from several manufacturers having the same formulation as VAZCULEP™ except having a different pH range of 3.0 to 6.5 or not including sodium metabisulfite.

BIORPHEN™ injection is available in the United States as a sterile solution in a glass ampoule containing 0.1 mg/mL or 10 mg/mL phenylephrine hydrochloride (equivalent to 0.08 mg/mL or 8.2 mg/mL phenylephrine base) and 9 mg/mL or 6.0 mg/mL sodium chloride in water for injection with sodium hydroxide and/or hydrochloric acid, if necessary, to provide a pH of 3.0 to 5.0. The prescribing information states that BIORPHEN™ 0.1 mg/mL injection must not be diluted before administration and that BIORPHEN™ 10 mg/mL injection must be diluted in 5% dextrose injection, USP or 0.9% sodium chloride injection before administration.

Other liquid pharmaceutical compositions of phenylephrine have been described. For example, U.S. patent Ser. No. 11/213,480 discloses a ready-to-use intravenous injection solution of phenylephrine hydrochloride packaged into a vial, where the solution consists essentially of about 0.05-0.15 mg/mL phenylephrine hydrochloride, about 3-4 mg/mL sodium chloride, about 0.1-3 mg/mL edetate disodium, and water, and has a pH between 3 and 6.5 (or pH from 5 to 5.3).

US Patent Application Publication 2021/0228507 discloses a ready-to-administer antioxidant free composition comprising not more than or equal to 0.4 mg/mL phenylephrine hydrochloride, an acetate buffer, and water, and has a pH between 4.5 and 5.5. The '507 publication discloses that when acetate was used as a buffering agent, the stability of low concentration phenylephrine was significantly increased as compared to a citrate or tartrate buffer with everything else being equal. The '507 publication discloses that the phenylephrine composition is packaged in a container, preferably a polymeric flexible bag for infusion or in a blow-fill-seal container.

US Patent Application Publication 2014/0235691 discloses a liquid pharmaceutical formulation for topical administration to the eye which comprises 30 μM to 720 μM phenylephrine and 10 μM to 270 μM ketorolac as the active pharmaceutical ingredients, 10 mM to 100 mM of a citrate buffer, and water, and has a pH of from 5.8 to 6.8, or preferably has a pH of about 6.3.

US Patent Application Publication 2017/0368124 discloses a composition comprising at least one herbal extract, a phenylephrine pharmaceutical composition, an acetate buffer and/or citric acid to maintain the pH of the herbal combination composition around 5.0.

There remains a need in the art for improved pharmaceutical formulations of phenylephrine which are ready-to-administer without dilution and stable, preferably following storage for long durations at room temperature.

### BRIEF SUMMARY OF THE INVENTION

The invention provides a formulation comprising about 0.010 mg/mL to about 0.20 mg/mL phenylephrine or a pharmaceutically acceptable salt thereof, about 0.5 mM to about 2 mM of a citrate buffer, a tonicity agent in an amount sufficient to provide the formulation with an osmolality of about 270 mOsm/kg to about 330 mOsm/kg, and water, wherein the formulation has an initial pH of from about 5.6 to about 6.0.

The invention also provides a formulation comprising about 0.10 mg/mL phenylephrine hydrochloride, about 2 mM to about 5 mM of a citrate buffer, about 0.1 to about 0.5 mg/mL sodium metabisulfite, about 8.0 to about 8.6 mg/mL sodium chloride, and water, wherein the formulation has an initial pH of from about 3.6 to about 4.8, or from about 5.5 to about 6.0.

### DETAILED DESCRIPTION OF THE INVENTION

The invention is based, at least in part, on the discovery of a phenylephrine formulation that is improved with respect to product stability (e.g., pH drift, impurity profile, and/or residual oxygen content) and attributes that contribute to patient compliance (e.g., reduced pain upon injection). The formulation of the invention preferably is ready-to-administer without dilution and stable, preferably following storage for long durations at room temperature.

The invention provides a formulation comprising about 0.010 mg/mL to about 0.20 mg/mL phenylephrine or a pharmaceutically acceptable salt thereof, about 0.5 mM to about 2 mM of a citrate buffer, a tonicity agent in an amount sufficient to provide the formulation with an osmolality of about 270 mOsm/kg to about 330 mOsm/kg, and water, wherein the formulation has an initial pH of from about 5.6 to about 6.0. The invention also provides a formulation comprising about 0.10 mg/mL phenylephrine hydrochloride, about 1 mM of a citrate buffer, a tonicity agent in an amount sufficient to provide the formulation with an osmolality of about 280 mOsm to about 300 mOsm, and water, wherein the formulation has an initial pH of about 5.8. The invention further provides invention also provides a formulation comprising about 0.10 mg/mL phenylephrine hydrochloride, about 2 mM to about 5 mM of a citrate buffer, about 0.1 to about 0.5 mg/mL sodium metabisulfite, about 8.0 to about 8.6 mg/mL sodium chloride, and water, wherein the formulation has an initial pH of from about 3.6 to about 4.8, or from about 5.5 to about 6.0.

As used herein, a “ready-to-administer” formulation refers to a sterile, injectable solution that need not be reconstituted from a solid or diluted from a concentrated solution by a healthcare provider prior to use. Rather, in the context of phenylephrine formulations of the invention, a ready-to-administer formulation is supplied by a pharmaceutical manufacturer as a liquid having a pharmaceutically effective amount of phenylephrine dissolved therein and contained within a suitable container (e.g., vial, syringe, bag, container, or the like) in a form that is intended to be administered to a subject without prior dilution by a healthcare provider.

As used herein, the term “initial pH” refers to the pH of a phenylephrine formulation at the end of a compounding process and prior to sterilization (e.g., aseptic filtration and/or thermal sterilization). The initial pH can be measured in a bulk phenylephrine formulation prior to filling into an individual container (e.g., syringe), or the initial pH can be measured in a phenylephrine formulation which has been filled into an individual container.

The formulation according to the present invention is stable. As used herein, the terms “stable” and “stability” encompass any characteristic of the formulation which may be affected by storage conditions including, without limitation, potency, total impurities, phenylephrine degradation products, specific optical rotation, optical purity, appearance, viscosity, sterility, particulates (visible and subvisible), color and/or clarity. The storage conditions which may affect stability include, for example, duration, temperature, humidity, and/or light exposure.

In some embodiments, the formulation of the invention is stable for at least about 12 months, e.g., at least about 18 months, at least about 24 months, or at least about 36 months, at room temperature (e.g., at 25° C.±2° C./60% RH±5% RH). In other embodiments, the formulation of the invention is stable for at least about 1 month, e.g., at least about 3 months, at least about 6 months, or at least about 12 months, under accelerated conditions (e.g., at 40° C.±2° C./75% RH±5% RH or 60° C.±2° C./75% RH±5% RH).

Methods for determining the stability of a formulation of the invention with respect to a given parameter are well-known in the art. For example, individual impurities and total impurities may be assessed by high-performance liquid chromatography (HPLC) or thin layer chromatography (TLC). Unless indicated otherwise, a percentage amount of any individual impurity or total impurities reported herein in the formulation is determined by a peak area percent method using HPLC.

In some embodiments, a stable phenylephrine formulation may refer to a formulation that is colorless after storage under room temperature and/or accelerated conditions. The color of the formulation may be determined, for example, by a United States Pharmacopoeia (USP) or European Pharmacopoeia (Ph. Eur.) color method. For example, a stable phenylephrine formulation of the invention may refer to a formulation that has a coloration of not more than B8 as determined by Ph. Eur. Method 2.2.2 after storage for at least 6 months at room temperature. By way of further example, a stable phenylephrine formulation of the invention may refer to a formulation that has a coloration of not more than B8 as determined by Ph. Eur. Method 2.2.2 after storage for at least 1 month under accelerated conditions (e.g., at 40° C.±2° C./75% RH±5% RH or 60° C.±2° C./75% RH±5% RH).

In other embodiments, a stable phenylephrine formulation may refer to a formulation that contains at least about 90%, e.g., least about 95%, at least about 96%, at least about 97%, at least about 98%, or at least about 99%, of the labeled concentration of phenylephrine or pharmaceutically acceptable salt thereof after storage under room temperature (e.g., 25° C.±2° C./60% relative humidity (RH)+5% RH) and/or accelerated (e.g., at 40° C.±2° C./75% RH±5% RH or 60° C.±2° C./75% RH±5% RH) conditions. A stable phenylephrine formulation also may refer to a formulation that contains not more than about 110%, e.g., not more than about 105%, not more than about 104%, not more than about 103%, not more than about 102%, or not more than about 101%, of the labeled concentration of phenylephrine or pharmaceutically acceptable salt thereof after storage under room temperature and/or accelerated conditions. A stable phenylephrine formulation additionally may refer to a formulation that contains from about 95% to about 105%, from about 96% to about 104%, from about 97% to about 103%, from about 98% to about 102%, or from about 99% to about 101%, of the labeled concentration of phenylephrine or pharmaceutically acceptable salt thereof after storage under room temperature and/or accelerated conditions.

A stable phenylephrine formulation also may refer to a formulation that contains not more than about 5%. e.g., not more than about 4%, not more than about 3%, not more than about 2%, not more than about 1%, or not more than 0.5% of total phenylephrine-related impurities present in the formulation after storage under room temperature and/or accelerated conditions. A stable phenylephrine formulation additionally may refer to a formulation that contains from about 0.1% to about 5%, e.g., from about 0.3% to about 3%, from about 0.4% to about 2.5%, from about 0.2% to about 1.2%, or from about 0.5% to about 2%, of total phenylephrine-related impurities present in the formulation after storage under room temperature and/or accelerated conditions.

A stable phenylephrine formulation also may refer to a formulation that contains not more than about 1%. e.g., not more than about 0.8%, not more than about 0.4%, not more than about 0.2%, or not more than about 0.1%, of any individual phenylephrine-related impurity present in the formulation after storage under room temperature and/or accelerated conditions. A stable phenylephrine formulation additionally may refer to a formulation that contains from about 0.05% to about 0.4%, e.g., from about 0.1% to about 0.3%, from about 0.05% to about 0.2%, or from about 0.2% to about 0.6%, of any individual phenylephrine-related impurity present in the formulation after storage under room temperature and/or accelerated conditions.

It has been surprisingly discovered that the phenylephrine formulation of the invention is particularly stable with respect to Impurity 9, which is a citrate adduct of phenylephrine having the following chemical structure

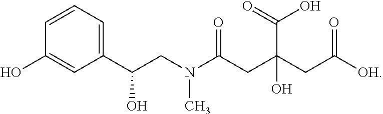 [SMILES: CN(C[C@H](O)c1cccc(O)c1)C(=O)CC(O)(CC(=O)O)C(=O)O]

In certain embodiments, the stable formulation contains not more than about 0.5%, e.g., not more than about 0.4%, not more than about 0.3%, not more than about 0.2%, or not more than about 0.1%, or not more than about 0.05% of Impurity 9 after storage under room temperature and/or accelerated conditions. A stable phenylephrine formulation additionally may refer to a formulation that contains from about 0.01% to about 0.5%, e.g., from about 0.05% to about 0.2%, from about 0.05% to about 0.1%, or from about 0.1% to about 0.3%, of Impurity 9 after storage under room temperature and/or accelerated conditions.

The formulation may include a therapeutically effective amount of phenylephrine or a pharmaceutically acceptable salt thereof. In some embodiments, the formulation of the invention includes a therapeutically effective amount of phenylephrine hydrochloride. The amount of phenylephrine or pharmaceutically acceptable salt thereof may be at a concentration of, e.g., from about 0.01 mg/mL to about 1 mg/mL, e.g., from about 0.05 mg/mL to about 0.20 mg/mL, about 0.010 mg/mL to about 0.20 mg/mL, or, e.g., about 0.10 mg/mL. In some embodiments, the formulation includes from about 0.01 mg/ml to about 1 mg/ml, e.g., from about 0.05 mg/ml to about 0.20 mg/ml or about 0.010 mg/mL to about 0.20 mg/mL, of phenylephrine hydrochloride. In certain embodiments, the formulation includes about 0.10 mg/mL phenylephrine hydrochloride, which corresponds to about 0.082 mg/mL phenylephrine base.

The formulation may be provided in any suitable volume. In some embodiments, the volume of the formulation is about 1 mL or more, e.g., about 2 mL or more, about 3 mL or more, about 5 mL or more, about 8 mL or more, about 10 mL or more, about 20 mL or more, or about 50 mL or more. The formulation also may be provided in a volume of about 100 mL or less, e.g., about 80 mL or less, about 60 mL or less, about 40 mL or less, about 30 mL or less, about 15 mL or less, about 10 mL or less, or about 5 mL or less. The formulation additionally may be provided in a volume bounded by any two of the aforementioned endpoints. For example, the formulation may be provided in a volume of from about 1 mL to about 100 mL, e.g., from about 3 mL to about 60 mL, from about 5 mL to about 30 mL, or from about 8 mL to about 15 mL. Preferably, the formulation is provided in a volume of 3 mL to 15 mL, more preferably in a volume of 5 mL to 10 mL. In some embodiments, the volume of the formulation is about 5 mL. In other embodiments, the volume of the formulation is about 10 mL. One of ordinary skill in the art may readily select an appropriate container based upon the volume of the formulation.

The formulation of the invention may include at least one tonicity agent. Suitable tonicity agents may include, without limitation, sodium chloride, dextrose, mannitol, trehalose, potassium chloride, glycerol, and combinations thereof. In some embodiments, the tonicity agent is sodium chloride or dextrose. In some embodiments, the tonicity agent is sodium chloride. The tonicity agent may present in an amount that renders the formulation isotonic. In some embodiments, the tonicity agent is present in an amount sufficient to provide the formulation with an osmotic pressure of about 250-350 mOsm/kg, e.g., about 270-330 mOsm/kg, about 260-320 mOsm/kg, about 300-340 mOsm/kg, or about 280-300 mOsm/kg. For example, the tonicity agent may be present in an amount sufficient to provide the formulation with an osmotic pressure of about 290 mOsm/kg. The invention also includes embodiments in which the formulation includes about 8 mg/mL to about 9 mg/mL sodium chloride. In certain embodiments, the formulation includes about 8.3 mg/mL sodium chloride.

The formulation of the invention also may include at least one buffer. The type and amount of buffer present in the formulation may be selected based on several considerations, including but not limited to, a target pH, pH stabilization, impurity formation, coloration, and/or patient tolerance upon administration. In some embodiments, the buffer may include a weak acid and a conjugate base of the weak acid. The weak acid and conjugate base may be added to the formulation in an anhydrous or hydrated form. In some embodiments, the conjugate base may be present in salt form. In some embodiments, the acid or weak acid component may include a dicarboxylic acid or a tricarboxylic acid. For example, the acid component may include citric acid, isocitric acid, aconitic acid, trimesic acid, propane-1,2,3-tricarboxylic acid, fumaric acid, oxalic acid, maleic acid, malonic acid, glutaric acid, succinic acid, tartaric acid, or a combination thereof. In some embodiments, the buffer includes citric acid and a salt thereof (i.e., a citrate salt). In certain embodiments, the buffer includes citric acid monohydrate and sodium citrate dihydrate. In other embodiments, the buffer includes anhydrous citric acid and trisodium citrate dihydrate. The invention also includes embodiments in which the buffer does not include acetic acid or an acetate salt. Such embodiments include a formulation of the invention which is substantially free of acetic acid and acetate salts.

The amount of buffer in the formulation can be selected based upon the forgoing considerations, e.g., target initial pH, pH stabilization, impurity formation, coloration, and/or patient tolerance upon administration. In some embodiments, the buffer may be present at a concentration of about 20 mM or less, e.g., about 10 mM or less, about 8 mM or less, about 6 mM or less, about 4 mM or less, or about 2 mM or less. The buffer also may be present at a concentration of about 0.1 mM or more, e.g., about 0.3 mM or more, about 0.5 mM or more, about 0.6 mM or more, about 0.8 mM, or about 1 mM or more. In some embodiments, the buffer is present at a concentration of about 0.1 mM to about 20 mM. e.g., about 0.2 mM to about 10 mM, about 0.5 mM to about 5 mM, about 0.5 mM to about 2 mM, about 1 mM to about 3 mM, about 2 mM to about 5 mM, or about 0.8 mM to about 2.5 mM. In certain embodiments, the buffer includes a citrate buffer which is present at a concentration of about 0.1 mM to about 20 mM, e.g., about 0.2 mM to about 10 mM, about 0.5 mM to about 5 mM, about 0.5 mM to about 2 mM, about 1 mM to about 3 mM, about 2 mM to about 5 mM, or about 0.8 mM to about 2.5 mM. In some embodiments, the formulation includes about 1 mM, about 2 mM, about 2.5 mM, about 3 mM, about 3.5 mM, about 4 mM, about 4.5 mM, or about 5 mM of a citrate buffer.

The weight or molar ratio of the weak acid to conjugate base of the buffer can be about 1 or less, e.g., about 0.8 or less, about 0.6 or less, about 0.4 or less, about 0.3 or less, or about 0.2 or less. The weight or molar ratio of the weak acid to conjugate base of the buffer also may be about 0.05 or more, e.g., about 0.1 or more, about 0.2 or more, about 0.3 or more, about 0.4 or more, or about 0.6 or more. The invention also includes embodiments in which the molar ratio of the weak acid to conjugate base is in a range bounded by the above endpoints, e.g., wherein the ratio is in the range of about 0.05 to about 1, e.g., about 0.1 to about 0.6, about 0.2 to about 0.4, about 0.3 to about 0.5, or about 0.2 to about 0.3. In some embodiments, the formulation comprises citric acid monohydrate and sodium citrate dihydrate present in a weight ratio of about 0.25.

The formulation may have an initial pH of greater than about 5.5, e.g., greater than about 5.6, greater than about 5.7, greater than about 5.8, greater than about 5.9, or greater than about 6.0. The formulation may have an initial pH of not more than about 6.5, e.g., not more than about 6.4, not more than about 6.3, not more than about 6.2, not more than about 6.1, not more than about 6.0, or not more than about 5.9. In some embodiments, the formulation has an initial pH of about 5.5 to about 6.5, e.g., about 5.6 to about 6.4, about 5.7 to about 6.3, about 5.8 to about 6.2, or about 5.6 to about 6.0. In certain embodiments, the formulation has an initial pH of about 5.6, about 5.7, about 5.8, about 5.9, about 6.0, about 6.1, or about 6.2.

In other embodiments, the formulation may have an initial pH of less than about 5.0, e.g., less than about 4.8, less than about 4.6, less than about 4.4, less than about 4.3, less than about 4.2, or less than about 4.1. The formulation may have an initial pH of more than about 3.4, e.g., more than about 3.5, more than about 3.6, more than about 3.7, more than about 3.8, or more than about 3.9. In some embodiments, the formulation has an initial pH of about 3.4 to about 5.0, e.g., about 3.6 to about 4.8, about 3.7 to about 4.6, about 3.8 to about 4.4, or about 3.9 to about 4.1. In certain embodiments, the formulation has an initial pH of about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0.

The formulation of the invention advantageously exhibits pH stability as evidenced by low pH drift following storage under room temperature and/or accelerated conditions. As used herein, the term “pH drift” refers to the amount of a change in pH of a formulation from a first pH, e.g., an initial pH, to a second pH, e.g., after thermal sterilization and/or storage. In some embodiments, the pH drift of the formulation is not more than about 0.4 pH units, e.g., not more than about 0.3 pH units, not more than about 0.25 pH units, not more than about 0.2 pH units, not more than about 0.15 pH units, not more than about 0.1 pH units, or not more than about 0.05 pH units, following storage for at least 6 months at room temperature. The invention also includes embodiments in which the pH drift of the formulation is not more than about 0.4 pH units, e.g., not more than about 0.3 pH units, not more than about 0.25 pH units, not more than about 0.2 pH units, not more than about 0.15 pH units, not more than about 0.1 pH units, or not more than about 0.05 pH units, following storage for at least 12 months at room temperature. For example, the invention includes embodiments in which the pH drift of the formulation is not more than about 0.4 pH units, e.g., not more than about 0.3 pH units, not more than about 0.25 pH units, not more than about 0.2 pH units, not more than about 0.15 pH units, not more than about 0.1 pH units, or not more than about 0.05 pH units, following storage for at least 24 months at room temperature.

The invention additionally includes embodiments in which the pH drift of the formulation is not more than about 0.4 pH units, e.g., not more than about 0.3 pH units, not more than about 0.25 pH units, not more than about 0.2 pH units, not more than about 0.15 pH units, not more than about 0.1 pH units, or not more than about 0.05 pH units, following storage for at least 1 month under accelerated conditions. For instance, the invention includes embodiments in which the pH drift of the formulation is not more than about 0.4 pH units, e.g., not more than about 0.3 pH units, not more than about 0.25 pH units, not more than about 0.2 pH units, not more than about 0.15 pH units, not more than about 0.1 pH units, or not more than about 0.05 pH units, following storage for at least 3 months under accelerated conditions. The invention also includes embodiments in which the pH drift of the formulation is not more than about 0.4 pH units, e.g., not more than about 0.3 pH units, not more than about 0.25 pH units, not more than about 0.2 pH units, not more than about 0.15 pH units, not more than about 0.1 pH units, or not more than about 0.05 pH units, following storage for at least 6 months under accelerated conditions.

The invention accordingly provides, for example, a formulation comprising about 0.010 mg/mL to about 0.20 mg/mL phenylephrine hydrochloride, about 0.5 mM to about 2 mM of a citrate buffer, a tonicity agent in an amount sufficient to provide the formulation with an osmolality of about 270 mOsm to about 330 mOsm, and water, wherein the formulation has an initial pH of from about 5.6 to about 6.0, wherein the pH drift of the formulation is not more than about 0.2 pH units following storage for at least 3 months under accelerated conditions.

The invention also provides, for example, a formulation comprising about 0.010 mg/mL to about 0.20 mg/mL phenylephrine hydrochloride, about 0.5 mM to about 2 mM of a citrate buffer, a tonicity agent in an amount sufficient to provide the formulation with an osmolality of about 270 mOsm to about 330 mOsm, and water, wherein the formulation has an initial pH of from about 5.6 to about 6.0, wherein the pH drift of the formulation is not more than about 0.2 pH units following storage for at least about 6 months at room temperature, e.g., at least about 12 months at room temperature, at least 18 months at room temperature, or at least about 24 months at room temperature.

The formulation of the invention may further include one or more other substances. Non-limiting examples of such other substances may include, for example, one or more diluents, salts, stabilizers, solubilizers, antioxidants, preservatives, and the like, and combinations thereof.

In some embodiments, the formulation of the invention is substantially free of a metal ion chelator and/or antioxidant distinct or separate from the citrate buffer, thereby advantageously avoiding the need to include and administer such additives by injection. The invention accordingly includes embodiments in which the formulation is substantially free of, for example, a sulfite or a bisulfite. The invention also includes embodiments in which the formulation is substantially free of, e.g., an aminopolycarboxylic acid such as, for example, ethylenediaminetetraacetic acid (EDTA), ethylene glycol-bis(β-aminoethyl ether)-N,N,N′,N′-tetraacetic acid (EGTA), or a salt thereof. The invention further includes embodiments in which the formulation is substantially free of, e.g., butylated hydroxyl anisole (BHA), butylated hydroxyl toluene (BHT), ascorbic acid, alpha-tocopherol, or propyl gallate. The term “substantially free” as used herein with respect to an excipient means that no amount of the excipient is added during manufacture of the formulation and, if present, only may be present as an impurity. In some embodiments, the formulation does not contain a chelating agent.

In other embodiments, the formulation of the invention may include at least one antioxidant. The antioxidant can be selected from the group consisting of a sulfite, an amino acid, a vitamin E compound, BHA, BHT, ascorbic acid, propyl gallate, and pharmaceutically acceptable salts thereof. In certain embodiments, the antioxidant is sodium metabisulfite. One of ordinary skill in the art will understand that the amount of antioxidant necessary to suppress the formation of oxidative degradation products will vary based upon various factors, including the selected antioxidant, degree of oxygen protection during manufacture, exposure to metal (e.g., stainless steel) during manufacture, final oxygen content in primary container, and secondary packaging. In certain embodiments, the formulation of the invention that contains at least one antioxidant does not contain a chelating agent.

In certain embodiments, the formulation contains sodium metabisulfite in an amount of about 0.05 mg/mL or more, e.g., about 0.1 mg/mL or more, about 0.2 mg/mL or more, about 0.3 mg/mL or more, about 0.4 mg/mL or more, or about 0.5 mg/ml or more. In other embodiments, the formulation contains sodium metabisulfite in an amount of about 1.0 mg/mL or less, e.g., about 0.9 mg/mL or less, about 0.8 mg/mL or less, about 0.7 mg/mL or less, about 0.6 mg/mL or less, about 0.5 mg/mL or less, or about 0.4 mg/mL or less. In yet other embodiments, the formulation contains sodium metabisulfite in a range of about 0.05 mg/mL to about 1.0 mg/mL, e.g., about 0.1 mg/mL to about to about 0.8 mg/mL, about 0.2 mg/mL to about 0.6 mg/mL, or about 0.3 mg/mL to about 0.5 mg/mL. In certain embodiments, the formulation contains about 0.1 mg/mL, about 0.2 mg/mL, about 0.3 mg/mL, or about 0.4 mg/mL of sodium metabisulfite.

The formulation of the invention has excellent storage stability when stored in a pharmaceutical container. In some embodiments, the container includes a syringe, a cartridge, a vial, an ampoule, a bag, or a bottle. Preferably, the container includes a syringe or a vial.

The container may include a material such as, e.g., glass or a synthetic polymer. The synthetic polymer may include an organic polymer such as, for example, an organic polymer which includes a polyethylene, a polypropylene, a cyclic olefin polymer (COP), or a cyclic olefin copolymer (COC). In some embodiments, the container includes COC. One or more surfaces of the container may be treated with a compound, e.g., to limit reactivity with one or more components of the formulation of the invention. For example, the container may be treated with a silicone. By way of further example, the container may be treated with a sulfur-containing compound, e.g., ammonium sulfate. However, the invention also includes embodiments in which the container is not treated.

In some embodiments, the container includes a syringe. The syringe barrel may include or be made of, e.g., glass or plastic. A suitable plastic syringe may include a syringe barrel which includes or is made of an organic polymer such as, e.g., a polyethylene, a polypropylene, COP, or COC. In some embodiments, the plastic syringe barrel may include COC. The invention also includes embodiments in which the plastic syringe barrel includes an amorphous COC that is copolymerized from norbornene and ethylene using a metallocene catalyst, such as, e.g., TOPAS™ COC manufactured by Topas Advanced Polymers GmbH. Non-limiting examples of syringes suitable for use in the present invention are described in US Patent Application Publication No. 2015/0273133 which is incorporated herein by reference in its entirety.

The invention also includes embodiments in which the container includes a vial. The vial may be made of any suitable material, which may include, for example, glass or plastic. The glass vial may include, e.g., a transparent glass vial or a light protective glass vial.

The container for use with the formulation of the invention can be obtained from a manufacturer as an unsterile or a sterile product. The present invention is based, at least in part, on the surprising and unexpected discovery that the method of container sterilization can impact the stability of the formulation of the invention. In some embodiments, the container is sterilized by radiation-based (e.g., gamma, X-ray, or e-beam) technology. In other embodiments, the container is sterilized by gas-based (e.g., ethylene oxide) technology.

In some embodiments, the pharmaceutical container comprising a formulation of the invention is packaged within a secondary package. In certain embodiments, the secondary package is a pouch, blister, flow wrapper, tray, or bag. The secondary package can be comprised of an oxygen, light, and/or moisture barrier material, such as high density polyethylene (HDPE), ethylenevinyl alcohol (EVOH), polypropylene (PP), polyethylene terephthalate (PET), polyethylene naphthalate (PEN), and polyamide (PA), metalized film, aluminum foil, oxide coated films, and combinations thereof. In certain embodiments, the secondary package also comprises an oxygen absorber. The oxygen absorber can be a sachet, pouch, canister, capsule, sticker, or strip that is placed inside of the secondary package. Alternatively, or additionally, the oxygen absorber can be incorporated into the material of the secondary package. In some embodiments, the oxygen absorber is selected from the group consisting of reduced iron compounds, catechol, ascorbic acid and analogs thereof, metal ligands, unsaturated hydrocarbons, and polyamides.

The invention accordingly provides a container comprising a pharmaceutical formulation comprising an active pharmaceutical ingredient (API), a liquid carrier, an optional excipient, wherein the container is sterilized by ethylene oxide. In some embodiments, the API is selected from the group consisting of an opioid, benzodiazepine, a2-adrenergic receptor agonist, beta blocker, morphine, hydromorphone, hydrocodone, oxycodone, oxymorphone, codeine, buprenorphine, naloxone, naltrexone, fentanyl, remifentanil, sufentanil, alfentanil, meperidine, rocuronium, vecuronium, midazolam, lorazepam, diazepam, neostigmine, atropine, glycopyrrolate, dexmedetomidine, cisastracurium, ropivacaine, lidocaine, propofol, ketamine, succinylcholine, moxifloxacin, linezolid, levofloxacin, levetiracetam, vancomycin, cefepime, aztreonam, cefoxitin, ceftriaxone, cefazolin, cefotaxime, ceftazidime, gentamicin, oxacillin, nafcillin, penicillin, cefuroxime, ticarcillin, clavulanic acid, piperacillin, tazobactam, azithromycin, meropenem, ertapenem, tigecycline, micafungin, metronidazole, fluconazole, itraconazole, posaconazole, heparin, enoxaparin, dalteparin, theophylline, acetaminophen (paracetamol), ibuprofen, acetylcysteine, decitabine, azacitidine, docetaxel, pemetrexed, palonosetron, aprepitant, fosaprepitant, famotidine, amiodarone, nitroglycerin, nicardipine, clevidipine, dobutamine, esmolol, labetalol, metroprolol, somatropin, liraglutide, abaloparatide, semaglutide, teriparatide, degarelix, sumatriptan, epinephrine, ephedrine, vasopressin, methotrexate, testosterone, and hydroxyprogesterone. In certain embodiments, the API is phenylephrine or a pharmaceutically acceptable salt thereof. In other embodiments, the API is atropine or a pharmaceutically acceptable salt thereof.

The pharmaceutical container may be sealed, e.g., by way of a closure, such as, e.g., a stopper, valve, plunger, and/or tip cap. In some embodiments, the closure may include an inert material such as, e.g., rubber or plastic. The closure also may be coated. e.g., with a silicone polymer or a fluoropolymer. However, the invention also includes embodiments which include a container in which the closure is not coated. Not limiting examples of materials that may be used in suitable closures include, for example, bromobutyl rubber, chlorobutyl rubber, and coated versions thereof.

The invention accordingly provides, for example, a syringe that includes the stable, ready-to-administer phenylephrine formulation described herein, wherein the syringe barrel includes a cyclic olefin copolymer, and the plunger includes uncoated bromobutyl rubber. In some embodiments, the syringe is sterilized by ethylene oxide.

A phenylephrine formulation of the invention may be prepared by any suitable technique, many of which are known in the art. The formulation may be prepared, e.g., in a batch or continuous process. In some embodiments, the formulation may be prepared by combining the components thereof in any order. The term “component” as used herein includes individual ingredients (e.g., phenylephrine or pharmaceutically acceptable salt thereof, tonicity agent, buffer, or water, etc.) as well as any combination of ingredients (e.g., phenylephrine or pharmaceutically acceptable salt thereof, tonicity agent, buffer, and/or water, etc.). In some embodiments, the formulation may be formed by combining the components together in a vessel. Such components may be combined in any order. The vessel may be made of any suitable material. In some embodiments, the vessel is made of or includes glass. In certain embodiments, the vessel is a glass vessel and contact of the formulation with any stainless steel component during manufacture is minimal or absent. In some embodiments, the formulation is manufactured using a single-use system comprised primarily of polymeric components. The manufacturing process may incorporate light protection and/or oxygen protection using methods known in the art. In some embodiments, the formulation may be prepared without light protection. The invention also includes embodiments in which the formulation is prepared without oxygen protection, and embodiments in which the formulation is prepared without light protection or oxygen protection.

In some embodiments, an amount of water equal to at least about 85% of the desired batch volume is added to a suitable vessel, then the tonicity agent and buffer are added, either sequentially or together, and the mixture is stirred until dissolution is complete. Subsequently, the phenylephrine or pharmaceutically acceptable salt thereof is added, and the mixture is stirred until dissolution is complete. Next, the pH is checked and adjusted, if necessary, by addition of an acid (e.g., hydrochloric acid) and/or a base (e.g., sodium hydroxide), to achieve the desired pH (e.g., about 5.6 to about 6.0 or about 3.6 to about 4.8). Alternatively, an acid or base pH adjuster is not added to the formulation. Next, an amount of water is added to bring the solution to the final desired batch volume. Optional ingredients, such as, e.g., one or more diluents, salts, stabilizers, solubilizers, antioxidants, and preservatives, may be provided to the formulation at any stage in its preparation.

The invention also includes embodiments in which the formulation is filtered, e.g., through one or more filters prior to filling the composition into one or more suitable containers, such as, e.g., a syringe, a vial, an ampoule, a cartridge, or a bag. In some embodiments, the container has been sterilized, e.g., by X-ray, gamma ray, or ethylene oxide, prior to filling. In certain embodiments, one or more filtration steps and the filling step are performed under aseptic conditions.

The filled containers may be subjected to a terminal sterilization process, such as, e.g., thermal sterilization. For example, thermal sterilization may be performed using water as a sterilizing medium. For sterilizing with water as the sterilization medium, the temperature of the water is preferably at least about 100° C., e.g., at least about 110° C., e.g., at least about 120° C. The thermal sterilization may be performed at a pressure of at least about 1 bar (100 kilopascal), for example, at least about 1.5 bar (150 kilopascal), at least about 1.7 bar (170 kilopascal), at least about 2 bar (200 kilopascal), at least about 3 bar (300 kilopascal), or at least about 4 bar (400 kilopascal). In some embodiments, thermal sterilization is performed at a pressure of from about 1 bar to about 4 bar. e.g., about 1-3 bar, about 1.5-2 bar, about 1.7-2 bar, or about 1.7-3 bar.

Thermal sterilization is may be carried out for at least about 10 minutes, e.g., for at least about 15 minutes, or for at least about 20 minutes.

In some embodiments, thermal sterilization of containers containing the phenylephrine formulation of the invention is carried out at a temperature of about 120° C.-122° C. and a pressure of about 2 bar (200 kilopascal) for about 15-20 minutes, for example, at a temperature of about 121° C. and a pressure of about 2 bar (200 kilopascal) for about 15 minutes.

The formulation according to the invention is suitable for administration to a subject to treat or prevent a disease or condition, including a disease or condition that is treatable with phenylephrine or a pharmaceutically acceptable salt thereof. Preferably, the subject is a mammal such as, for example, a human. The disease or condition that is treatable by the administration of phenylephrine or a pharmaceutically acceptable salt thereof may include, for example, hypotension. In some embodiments, the condition may include clinically important hypotension resulting primarily from vasodilation in the setting of anesthesia. The invention thus provides, for example, a method for treating hypotension including, e.g., hypotension during anesthesia, by administering a formulation as described herein to a patient in need thereof.

The following examples further illustrate the invention but, of course, should not be construed as in any way limiting its scope.

### Example 1

This example demonstrates the amounts of impurities present in aqueous formulations comprising phenylephrine hydrochloride, a tonicity agent and a citrate buffer having an initial pH of 4.5 or 5.5 packaged in plastic or glass syringes.

Samples containing 0.10 mg/mL phenylephrine hydrochloride and 0 mM, about 0.2 mM, or about 20 mM citrate buffer were prepared in a glass vessel, adjusted to pH of 4.5 or 5.5 with hydrochloric acid and/or sodium hydroxide and to osmolality of 280-320 mOsm/kg with sodium chloride, filled into unsterilized containers (plastic COC syringes, plastic COP syringes, or glass syringes), plugged with uncoated bromobutyl rubber stoppers, thermally sterilized at 121° C. for 15 minutes, and placed into a stability chamber under accelerated (60° C.±2° C./75% RH±5% RH) conditions. The amount of total impurities (percent amount of phenylephrine API) of each sample was determined by HPLC after 2 weeks of storage, and the results are shown in Table 1.

TABLE 1

| | COC | COP | Glass |
|---|---|---|---|
| Unbuffered, pH 4.5 | 1.77 | 2.34 | 0 |
| Unbuffered, pH 5.5 | 1.55 | 1.66 | 0.14 |
| 0.2 mM citrate, pH 4.5 | 3.9 | 3.5 | 0 |
| 0.2 mM citrate, pH 5.5 | 1.25 | 2.0 | 0 |
| 20 mM citrate, pH 4.5 | 3.85 | 4.65 | 1.7 |
| 18 mM citrate, pH 5.5 | 1.80 | 1.85 | 0.15 |

The results of this example demonstrate that the amount of total impurities in the tested samples was (i) higher when packaged in plastic syringes as compared to glass syringes, (ii) generally lower when packaged in COC syringes as compared to COP syringes, (iii) higher in citrate buffered samples as compared to unbuffered samples, and (iv) lower at pH 5.5 as compared to pH 4.5.

### Example 2

This example demonstrates the pH drift and the amounts of impurities present in aqueous formulations comprising phenylephrine hydrochloride, a tonicity agent and, a citrate buffer packaged in plastic syringes as a function of the pH and buffer concentration of the formulation and oxygen protection during manufacture.

Samples containing 0.10 mg/mL phenylephrine hydrochloride and 0 mM, about 0.2 mM, or about 2 mM citrate buffer were prepared in a glass vessel, adjusted to pH of 5.0, 6.0, or 6.5 with hydrochloric acid and/or sodium hydroxide and to osmolality of 280-320 mOsm/kg with sodium chloride, filled into unsterilized plastic COC syringes, plugged with uncoated bromobutyl rubber stoppers, thermally sterilized at 121° C. for 15 minutes, and placed into a stability chamber under accelerated (60° C.±2° C./75% RH±5% RH) conditions. A subset of the thermally sterilized, filled syringes at each pH and buffer concentration was packaged into an aluminum bag with an oxygen absorber.

The pH of each sample was determined after 2-weeks of storage under accelerated conditions, and the results are shown in Table 2.

TABLE 2

t = 2 W 60° C.

| Formulation | pH value | Unprotected | O₂-protect |
|---|---|---|---|
| Unbuffered | 5.0 | 4.9 | 5.7 |
| | 6.0 | 5.3 | 6.0 |
| | 6.5 | 5.5 | 6.0 |
| 0.2 mM citrate | 5.0 | 4.9 | 5.0 |
| | 6.0 | 6.0 | 6.1 |
| | 6.5 | 6.6 | 6.7 |
| 2 mM citrate | 5.0 | 4.9 | 4.9 |
| | 6.0 | 5.9 | 5.9 |
| | 6.5 | 6.5 | 6.5 |

Generally, the results in Table 2 demonstrate (i) considerable pH fluctuations in unbuffered samples, (ii) a slight pH drift towards more alkaline pH in the 0.2 mM citrate buffered samples having an initial pH 6.5. (iii) stable pH values in the 2 mM citrate buffered samples, and (iv) oxygen protection did not impact pH drift.

The amount of total impurities (percent amount of phenylephrine API) in the thermally sterilized samples was determined by HPLC at t=0 and after 2 weeks of storage, and the results are shown in Table 3.

TABLE 3

| | pH | t = 0 | | t = 2 W 60° C. | |
|---|---|---|---|---|---|
| Formulation | value | Unprotected | O₂-protect | Unprotected | O₂-protect |
| Unbuffered | 5.0 | 0.46 | 0.56 | 0.89 | 0.52 |
| | 6.0 | 0.50 | 0.51 | 0.58 | 0.54 |
| | 6.5 | 0.54 | 0.53 | 0.58 | 0.54 |
| 0.2 mM | 5.0 | 0.52 | 0.46 | 0.73 | 0.63 |
| citrate | 6.0 | 0.52 | 0.49 | 0.52 | 0.49 |
| | 6.5 | 0.53 | 0.45 | 0.54 | 0.51 |
| 2 mM | 5.0 | 0.45 | 0.39 | 0.71 | 0.44 |
| citrate | 6.0 | 0.54 | 0.44 | 0.56 | 0.44 |
| | 6.5 | 0.49 | 0.46 | 0.54 | 0.46 |

The results in Table 3 demonstrate that the amount of total impurities in the tested formulations was higher in samples having an initial pH of 5.0 as compared to pH 6.0 or 6.5 and that there was minimal impact of oxygen on impurity formation in citrate buffered samples at pH 6.0 or 6.5.

### Example 3

This example demonstrates the effect of terminal sterilization on the impurities present in aqueous formulations comprising phenylephrine hydrochloride, a tonicity agent and a citrate buffer packaged in plastic syringes.

Samples containing 0.10 mg/mL phenylephrine hydrochloride, 9 mg/mL sodium chloride, and about 0.2 mM citrate buffer were prepared in a glass vessel, adjusted to pH of 6.0 with hydrochloric acid and/or sodium hydroxide, filled into unsterilized plastic COC syringes, and plugged with uncoated bromobutyl rubber stoppers. Placebo samples were prepared similarly except for not containing phenylephrine API. Subsets of the phenylephrine and placebo samples were thermally sterilized by autoclave at 121° C. for 15 minutes. The samples were placed into a stability chamber under accelerated (60° C.±2° C./75% RH±5% RH) conditions, and the amounts of individual impurities in the samples were determined by HPLC at t=0 and after 2 weeks of storage. The results are shown in Table 4, where the individual impurities are provided as a percent of phenylephrine API in the phenylephrine samples.

TABLE 4

| | | t = 0 | | t = 2 W 60° C. | |
|---|---|---|---|---|---|
| Formulation | Impurity | No TS | TS | No TS | TS |
| Phenylephrine | RRT 0.37 | nd | nd | 0.10 | 0.10 |
| | RRT 0.77 | nd | nd | 0.12 | 0.13 |
| | RRT 0.88 | nd | 0.22 | 0.32 | 0.33 |
| | RRT 1.98 | nd | 0.40 | 0.21 | 0.18 |
| Placebo | RRT 0.37 | nd | nd | 0.10 | 0.10 |
| | RRT 0.77 | nd | nd | nd | nd |
| | RRT 0.88 | nd | nd | nd | nd |
| | RRT 1.98 | nd | 0.27 | 0.15 | 0.17 |

nd = not detected

The results of this example demonstrate that (i) no impurities are detected in non-terminally sterilized samples at t=0, (ii) two impurities (RRT 0.77 and RRT 0.88) are related to the phenylephrine API, (iii) two impurities (RRT 0.37 and RRT 1.98) are related to the container system, and (iv) the impurity profiles of non-terminally sterilized samples are similar to terminally sterilized samples after storage at 60° C. for two weeks.

### Example 4

This example demonstrates the stability of phenylephrine formulations as a function of the method used to sterilize the primary container and type of manufacturing vessel.

A batch containing 0.10 mg/mL phenylephrine hydrochloride, 9 mg/mL sodium chloride, 0.2 mM citrate buffer, adjusted to pH of 6.0 with hydrochloric acid and/or sodium hydroxide in water was prepared in a glass vessel, and then split into a glass bottle or a stainless steel tank. After incubation at 25° C. for 24 hours, samples were filled into plastic COC syringes that were not previously sterilized or were sterilized by gamma irradiation or by X-rays, plugged with uncoated bromobutyl rubber stoppers, thermally sterilized at 121° C. for 15 minutes, and placed into a stability chamber under accelerated (40° C.±2° C./75% RH±5% RH) conditions.

The amounts of individual impurities in the samples were determined by HPLC at t=0 and after 3 months of storage under accelerated conditions. The results are shown in Table 5, where the individual impurities are provided as a percent of phenylephrine API.

TABLE 5

| | | t = 0 | | t = 3 M 40° C. | |
|---|---|---|---|---|---|
| Syringe | Impurity | Glass | Stainless | Glass | Stainless |
| COC non- | RRT 0.77 | nd | nt | nd | nt |
| sterile | RRT 0.88 | nd | nt | nd | nt |
| | RRT 0.51 | nd | nt | nd | nt |
| COC gamma | RRT 0.77 | nd | nd | nd | 0.11 |
| | RRT 0.88 | 0.19 | 0.24 | 0.28 | 0.39 |
| | RRT 0.51 | nd | nd | nd | 0.11 |
| COC X-ray | RRT 0.77 | nd | nt | 0.12 | nt |
| | RRT 0.88 | 0.35 | nt | 0.45 | nt |
| | RRT 0.51 | nd | nt | nd | nt |

nd = not detected;
nt = not tested

The results in Table 5 demonstrate that that the level of impurities is higher in irradiated (gamma or X-ray) syringes as compared to non-sterile syringes and that pre-incubation of the phenylephrine formulation in a stainless steel vessel resulted in higher impurities.

### Example 5

This example demonstrates the stability of phenylephrine formulations in ethylene oxide (EtO) sterilized syringes as a function of the formulation buffer type.

Samples containing 0.10 mg/mL phenylephrine hydrochloride and 1 mM of citrate buffer or 1 mM acetate buffer in water adjusted to pH of 5.0 with hydrochloric acid and/or sodium hydroxide and to an osmolality of 280-300 mOsm/kg with sodium chloride were prepared in a stainless steel vessel with oxygen protection, and filled into plastic COC syringes that were sterilized by ethylene oxide treatment, plugged with uncoated bromobutyl rubber stoppers, packaged into an aluminum overpouch with an oxygen absorber, thermally sterilized, and then placed into a stability chamber under accelerated (40° C.±2° C./75% RH±5% RH) conditions.

The pH of each sample was determined at t=0 and after 1- and 3-months of storage under accelerated conditions, and the results are shown in Table 6.

TABLE 6

| Buffer | t = 0 | t = 1 M | t = 3 M |
|---|---|---|---|
| Citrate | 5.2 | 5.1 | 5.1 |
| Acetate | 6.1 | 5.3 | 5.6 |

The results in Table 6 demonstrate pH drift towards more acidic pH in 1 mM acetate buffered samples, which was not observed in 1 mM citrate buffered samples.

The amounts of individual impurities in the samples were determined by HPLC at t=0 and after 1 and 3 months of storage under accelerated conditions. The results are shown in Table 7, where the individual impurities are provided as a percent of phenylephrine API.

TABLE 7

| Buffer | Impurity | t = 0 | t = 1 M | t = 3 M |
|---|---|---|---|---|
| Citrate | RRT 0.35 | nd | nd | 0.12 |
| | RRT 0.43 | nd | nd | 0.06 |
| | RRT 0.89 | nd | nd | nd |
| | RRT 1.64 | 0.05 | 0.04 | nd |
| | Impurity 9 | nd | nd | nd |
| Acetate | RRT 0.35 | nd | nd | 0.11 |
| | RRT 0.43 | nd | nd | 0.05 |
| | RRT 0.89 | nd | 0.03 | 0.05 |
| | RRT 1.64 | 0.05 | 0.05 | nd |

nd = not detected

The results in Table 7 demonstrate generally similar levels of impurities in 1 mM citrate- or acetate-buffered phenylephrine formulations packaged in EtO-sterilized COC syringes. Impurity 9, which is a citrate adduct of phenylephrine that is known to form in commercially available, 18 mM citrate-buffered, phenylephrine HCl concentrate (10 mg/mL) liquid glass vial products, was not detected in 1 mM citrate-buffered phenylephrine formulations packaged in EtO-sterilized COC syringes.

### Example 6

This example demonstrates the stability of atropine formulations as a function of the method used to sterilize the primary container.

A batch containing 0.10 mg/mL atropine sulfate, 9 mg/mL sodium chloride, adjusted to pH of 3.5 with sulfuric acid and/or sodium hydroxide in water was prepared, filled into plastic COC syringes that were sterilized by gamma irradiation or by EtO, plugged with uncoated bromobutyl rubber stoppers, thermally sterilized at 121° C. for 15 minutes, and placed into a stability chamber under accelerated (40° C.±2° C./75% RH±5% RH) conditions.

The amounts of individual impurities in the samples were determined by HPLC at t=0 and after 1 and 3 months of storage under accelerated conditions. The results are shown in Table 8, where the individual impurities are provided as a percent of atropine API.

TABLE 8

| Syringe | Impurity | t = 0 | t = 1 M | t = 3 M |
|---|---|---|---|---|
| COC gamma | RRT 0.63 | nd | 0.42 | 0.61 |
| | RRT 0.67 | 0.09 | 0.47 | 0.73 |
| | RRT 0.73 | 0.09 | 0.48 | 0.76 |
| | Tropic acid | nd | 0.12 | 0.22 |
| | Apoatropine | 0.13 | 0.14 | 0.16 |
| COC EtO | RRT 0.63 | nd | 0.07 | nd |
| | RRT 0.67 | nd | 0.07 | 0.11 |
| | RRT 0.73 | nd | 0.08 | 0.13 |
| | Tropic acid | nd | 0.09 | 0.19 |
| | Apoatropine | 0.13 | 0.15 | 0.18 |

nd = not detected

The results in Table 8 demonstrate that the level of impurities in an atropine formulation is higher in gamma irradiated syringes as compared to EtO-sterilized syringes.

### Example 7

This example demonstrates the stability of phenylephrine formulations in plastic syringes as a function of pH and contents of and buffer and antioxidant.

Samples containing 0.10 mg/mL phenylephrine hydrochloride, sodium chloride, citrate buffer, and sodium metabisulfite adjusted to pH of about 4.0 or about 5.8 with hydrochloric acid and/or sodium hydroxide were prepared in a glass vessel with oxygen protection, filled into plastic COC syringes that were sterilized by X-ray treatment, and plugged with uncoated bromobutyl rubber stoppers. The headspace oxygen content of each sample was approximately 10%. The composition of each sample is described in Table 9.

TABLE 9

| Ingredient | Sample 1 | | Sample 2 | | Sample 3 | | Sample 4 | | Sample 5 | |
|---|---|---|---|---|---|---|---|---|---|---|
| Phenylephrine | 0.1 | mg / mL | 0.1 | mg / mL | 0.1 | mg / mL | 0.1 | mg / mL | 0.1 | mg / mL |
| HCl | | | | | | | | | | |
| Sodium Chloride | 8.3 | mg / mL | 8.3 | mg / mL | 8.3 | mg / mL | 8.3 | mg / mL | 8.3 | mg / mL |
| Sodium Citrate | 0.60 | mg/mL | 0.60 | mg/mL | 0.60 | mg/mL | 0.60 | mg/mL | 1.0 | mg/mL |
| Dihydrate | | | | | | | | | | |
| Citric Acid | 0.15 | mg/mL | 0.15 | mg/mL | 0.15 | mg/mL | 0.15 | mg/mL | 0.25 | mg/mL |
| Monohydrate | | | | | | | | | | |
| Sodium | 0.1 | mg/mL | 0.2 | mg/mL | 0.4 | mg/mL | 0.4 | mg/mL | 0.4 | mg/mL |
| Metabisulfite | | | | | | | | | | |

| | | | | | |
|---|---|---|---|---|---|
| NaOH / HCl | As needed | As needed | As needed | As needed | As needed |
| | for | for | for | for | for |
| | pH 4.0 | pH 4.0 | pH 4.0 | pH 5.8 | pH 4.0 |
| Purified Water | Q.S | Q.S | Q.S | Q.S | Q.S |

A subset of Sample 3 was contacted with stainless steel for 15 minutes prior to filling into syringes. Another subset of Sample 3 was secondarily packaged into an aluminum overpouch with an oxygen absorber. All samples were then placed into a stability chamber under room temperature (25° C.±2° C./40% RH±5% RH) or accelerated (40° C.±2° C./25% RH±5% RH) conditions. Samples of commercially available BIORPHEN™ ampules also were placed into the stability chamber for comparison.

Following storage for various durations (e.g., 2 weeks, 1 month, 2 months, 3 months, 4 months, 5 months, 6 months, 12 months, 18 months, or 24 months), the samples will be examined for various parameters (e.g., pH, potency/assay, total impurities, phenylephrine degradation products, specific optical rotation, optical purity, appearance, viscosity, sterility, particulates (visible and subvisible), color and/or clarity) using standard methodologies.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A formulation comprising about 0.010 mg/mL to about 0.20 mg/mL phenylephrine hydrochloride, about 0.5 mM to about 5 mM of a citrate buffer, a tonicity agent in an amount sufficient to provide the formulation with an osmolality of about 270 mOsm/kg to about 330 mOsm/kg, and water, wherein the formulation has an initial pH of from about 5.6 to about 6.0.

2. The formulation of claim 1, wherein the phenylephrine hydrochloride is present at a concentration of about 0.10 mg/mL.

3. The formulation of claim 1, wherein the tonicity agent comprises sodium chloride, dextrose, mannitol, trehalose, potassium chloride, glycerol, or a combination thereof.

4. The formulation of claim 1, wherein the formulation has an initial pH of about 5.8.

5. The formulation of claim 1, wherein the formulation comprises about 1 mM to about 3 mM of a citrate buffer.

6. The formulation of claim 1, wherein the formulation does not contain acetic acid or a salt thereof or ethylenediaminetetraacetic acid (EDTA) or a salt thereof, and the phenylephrine is the only active pharmaceutical ingredient in the formulation.

7. The formulation of claim 1, wherein the formulation has a pH drift of not more than about 0.2 pH units following storage for at least 3 months at 40° C.

8. The formulation of claim 1, wherein the formulation contains not more than about 0.2% total impurities as determined by a peak area percent method by high-performance liquid chromatography (HPLC) after storage for at least 3 months at 40° C.

9. The formulation of claim 1, wherein the formulation contains not more than about 0.05% of Impurity 9 as determined by a peak area percent method by high-performance liquid chromatography (HPLC).

10. A plastic container comprising the formulation of claim 1.

11. The plastic container of claim 10, wherein the container comprises a cyclic olefin polymer (COP) or a cyclic olefin copolymer (COC) and has been sterilized by X-ray treatment.

12. A formulation comprising about 0.10 mg/mL phenylephrine hydrochloride, about 1 mM to about 3 mM of a citrate buffer comprising citric acid monohydrate and sodium citrate dihydrate present in a weight ratio of about 0.25, a tonicity agent in an amount sufficient to provide the formulation with an osmolality of about 280 mOsm/kg to about 300 mOsm/kg, and water, wherein the formulation has an initial pH of about 5.8.

13. The formulation of claim 12, wherein the tonicity agent comprises about 8 mg/mL to about 9 mg/mL sodium chloride.

14. The formulation of claim 12, wherein the formulation does not contain acetic acid or a salt thereof or ethylenediaminetetraacetic acid (EDTA) or a salt thereof and the phenylephrine is the only active pharmaceutical ingredient in the formulation.

15. The formulation of claim 12, wherein the formulation has a pH drift of not more than about 0.2 pH units following storage for at least 3 months at 40° C.

16. The formulation of claim 12, wherein the formulation contains not more than about 0.2% total impurities as determined by a peak area percent method by high-performance liquid chromatography (HPLC) after storage for at least 3 months at 40° C.

17. The formulation of claim 12, wherein the formulation contains not more than about 0.05% of Impurity 9 as determined by a peak area percent method by high-performance liquid chromatography (HPLC).

18. A plastic container comprising the formulation of claim 12.

19. The plastic container of claim 18, wherein the container comprises a cyclic olefin polymer (COP) or a cyclic olefin copolymer (COC) and has been sterilized by X-ray treatment.

20. A pharmaceutical product comprising about 0.10 mg/mL phenylephrine hydrochloride, about 1 mM to about 3 mM of a citrate buffer comprising citric acid monohydrate and sodium citrate dihydrate present in a weight ratio of about 0.25, a tonicity agent in an amount sufficient to provide an osmolality of about 280 mOsm/kg to about 300 mOsm/kg, and water having an initial pH of 5.8 to provide a formulation, which is packaged in a plastic syringe that has been sterilized by X-ray treatment.

21. The pharmaceutical product of claim 20, wherein the formulation does not contain acetic acid or a salt thereof or ethylenediaminetetraacetic acid (EDTA) or a salt thereof and the phenylephrine is the only active pharmaceutical ingredient in the formulation.

22. The pharmaceutical product of claim 20, wherein, following storage of the product for at least 3 months at 40° C., the formulation has a pH drift of not more than about 0.2 pH units and contains not more than about 0.2% total impurities as determined by a peak area percent method by high-performance liquid chromatography (HPLC).

23. The formulation of claim 20, wherein the formulation contains not more than about 0.05% of Impurity 9 as determined by a peak area percent method by high-performance liquid chromatography (HPLC).

24-42. (canceled)
